(19) United States
(12) Patent Application Publication
(10) Pub. No.: US 2004/0244126 A1
(43) Pub. Date: Dec. 9, 2004

(54) Method, compositions, and kit for coloring hair

(76) Inventors: Lou Ann Christine Vena, Scotch Plains, NJ (US); Malti Vishin Bhambhani, Scotch Plains, NJ (US); Saroja Narasimhan, Matawan, NJ (US); Melissa Jamie Miczewski, Rahway, NJ (US); Shailendra Kumar Singh, Edison, NJ (US)

Correspondence Address: JULIE BLACKBURN, REVLON CONSUMER PRODUCTS CORPORATION, 237 PARK AVENUE, NEW YORK, NY, 10017, US

(21) Appl. No.: 10/454,405

(22) Filed: Jun. 4, 2003

(51) Int. Cl. A61K 7/135; A61K 7/13

(52) U.S. Cl. 8/406

(57) ABSTRACT

Methods, compositions, and kits for coloring hair and maintaining the color between oxidative dyeing procedures.

### TECHNICAL FIELD

The invention is in the field of compositions and methods for coloring hair, and kits containing the components necessary to practice the method and process.

### BACKGROUND OF THE INVENTION

It is estimated that about fifty percent of the female population colors their hair. In most cases the hair is colored to cover gray. In a smaller percentage of cases, the user simply desires to change the color of her hair.

Oxidative, or “permanent” hair color, which permanently changes the color of the hair, is most often used by consumers. While this type of hair color permanently changes the color of the hair, because hair grows about ¼ to ½ inch per month, new hair growth becomes evident at the hair roots in a month's time. Further, while oxidative hair color is permanent, in some cases the color may fade after a number of hair washings. This results in a noticeable color change, with some shades more vulnerable than others. For example, this phenomenon is more common in red shades; which may become muted and fade after only two weeks. It appears that that fading seen in oxidatively colored hair is partially due to contact of the hair with water in regular cleansing of the hair with shampoos. Secondarily, hair color fading may result when hair is exposed to sunlight or the elements.

There are a variety of products available that their manufacturers claim will ameliorate the fading of oxidatively colored hair. For example, U.S. Pat. No. 6,143,286 teaches a hair conditioner that contains a certain type of cross-linkable silicone that reacts with the hair shaft and thereby “locks in” color. Also known are hair conditioner compositions that contain semi-permanent dyes. Such conditioners are sold by Logics or Aveda, and are standard hair conditioners that contain various combinations of semi-permanent dyes. The consumer purchases such hair conditioners for use after shampooing, either in place of, or in addition to her normal hair conditioner. The semi-permanent dyes present will, to some extent, coat the oxidatively colored hair that has begun to fade and will provide a temporary color boost to improve the vibrancy and life of the underlying color. However, it is well known that semi-permanent dyes, and in particular the so-called cationic dyes, have medium to poor colorfastness on hair and a reputation for poor light resistance and uneven coloring of the hair between root and tip. In addition, the known cationic dyes have an insufficient build-up; i.e., even if increased amounts are used, it is impossible to exceed a certain, relatively low, color strength. For instance, it is not possible to achieve deep black coloration with the most important cationic hair dyes Basic Yellow 57, Basic Red 76, Basic Blue 99, Basic Brown 16 and Basic Brown 17 which are used in practice. Accordingly color conditioners containing cationic dyes are not always as effective as consumers desire.

Another common problem with such commercially available color conditioners is that they are not packaged in the retail hair color kit purchased by the consumer who colors her hair. In this situation, the consumer purchases a retail kit containing the oxidative dye composition, developer, and a standard hair conditioner and performs the hair color process. Thereafter, if the consumer desires to prolong the color, reduce the fade resistance, or improve color tone, he or she is relegated to hunting for these so-called color boosting shampoos or conditioners on drugstore shelves or in salons. In this situation, the product is not made for use with the compositions in the oxidative dye kit, and in the case where the consumer purchases a product such as a hair conditioner with semi-permanent dyes, she is left with the task of trying to figure out which conditioner provides the best match with her oxidatively colored hair. In cases where the color match is not optimal, the result can be a hair color that is not flattering or desired by the consumer. The selection of the appropriate hair conditioner is further complicated by the fact that exact color matching of the hair conditioner with the oxidative hair color shade does not always ensure a good end result. More than just color must be considered in selecting the appropriate conditioner. Particularly important is tonality, as well as dimensionality and hue, in achieving and maintaining the appearance of professionally colored hair between oxidative dye sessions.

Accordingly, there is a need for a simple, inexpensive compositions, methods, and kits to enable those who oxidatively color their hair to maintain the color in between oxidative coloring procedures. The components should be inexpensive to manufacture, and the color maintaining composition (or after care composition) should match with the oxidatively colored hair and be capable of returning it to its original hue, tone, and dimensionality. Moreover, the products should be storage stable and the after care composition (which can be in the form of a shampoo, conditioner, leave on composition, etc.) should be in consumer friendly package that is suitable for either single or multiple uses.

An object of the invention is to provide a method for improving the color deposition on, and fade resistance of, oxidatively dyeing hair by applying the after care composition onto the hair immediately after the oxidative dyeing procedure, and thereafter, if desired, at intermittent periods, for example every 1 to 30 days, or optimally every 7 to 10 days, until the next oxidative coloring procedure.

Another object of the invention is to provide compositions, including oxidative dye, developer, and after care compositions, that have ingredients present that maximize color deposition and minimize fade resistance.

Another object of the invention is to provide a kit for purchase by the retail consumer that contains all of the components necessary to oxidatively color hair along with the appropriate after care composition for use in maintaining the hair color, which after care composition is matched with the tonality, vibrancy, and hue of the oxidatively colored hair.

Another object of the invention is to provide a method and kit for improving the tonality of oxidatively colored hair, more specifically to provide certain aesthetic color tones to hair that has been oxidatively colored in order to provide more vibrant hair color with a multi-dimensional effect.

### SUMMARY OF THE INVENTION

The invention comprises a kit for oxidatively coloring hair and maintaining the color comprising, in combination:

(a) at least one oxidative composition,

(b) at least one developer composition operable to react with the oxidative composition when combined therewith, to form a mixture that will modify the color of the hair when applied thereto; and

(c) at least one aftercare composition comprising one or more semi-permanent dyes selected to complement the color of the hair oxidatively colored by the mixture of compositions (a) and (b).

The invention further comprises a method for improving color deposition and fade resistance of oxidatively colored hair by applying an after care composition containing at least one semi-permanent dye immediately after oxidatively coloring the hair, and repeating such applications of after care composition at intermittent time periods between oxidative dye sessions.

The invention further comprises a method for providing unique tones to oxidatively colored hair by applying an aftercare composition containing at least one semi-permanent dye to the hair, in a color and amount sufficient to provide unique color tones overlayered onto the color of the oxidatively dyed hair.

### DETAILED DESCRIPTION

I. The Kit

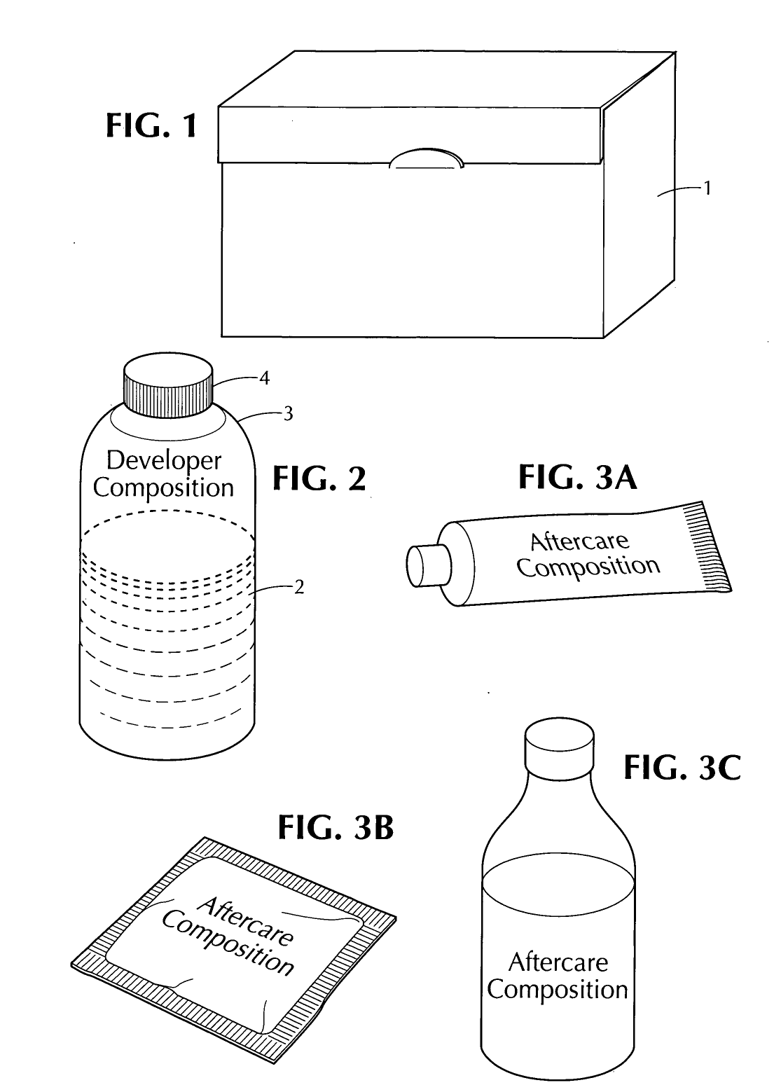

The invention comprises a kit for use in coloring hair and maintaining the color comprising the various components depicted in FIGS. 1-5.

FIG. 1: depicts a container for storing the various components of the kit including the oxidative composition, the developer composition, and the aftercare composition.

FIG. 2: depicts the developer composition stored in a receptacle with a cap.

FIG. 3: in general depicts the aftercare composition and examples of the different types of containers suitable for storing the aftercare composition in the kit.

FIG. 3a: depicts the aftercare composition stored in a tube container.

FIG. 3b: depicts the aftercare composition stored in a packette type container.

FIG. 3c: depicts the aftercare composition stored in a bottle.

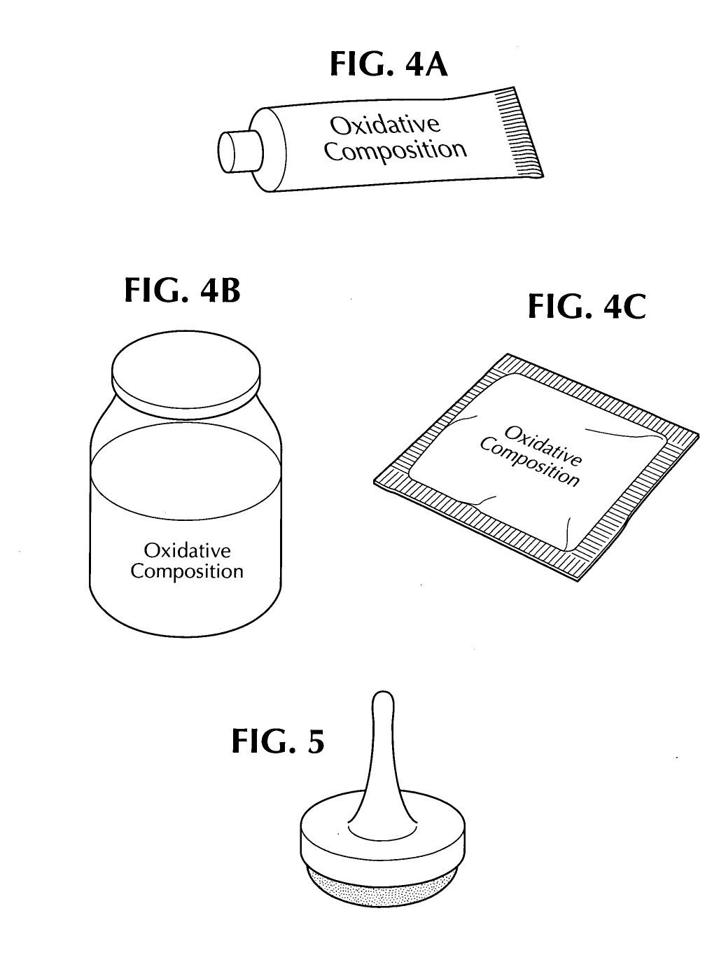

FIG. 4: in general depicts the oxidative composition and examples of the different types of containers suitable for storing the oxidative composition in the kit.

FIG. 4a: depicts the oxidative composition stored in a tube container.

FIG. 4b: depicts the oxidative composition stored in a jar.

FIG. 4c: depicts the oxidative composition stored in a packette.

FIG. 5: depicts an application nozzle for use in applying the mixture of the oxidative composition and the developer composition to the hair.

The container 1 for storing the various components of the kit may be made of cardboard, plastic, or any other suitable material so long as it is sturdy enough to withstand commercial requirements. Preferably the container 1 is made of cardboard and it may be printed with graphics, such as models wearing the hair color found within, a panel listing the ingredients found in each of the compositions, UPC codes, manufacturer information, and the like.

The developer composition 2 is most preferably stored in a bottle 3 that is made from some thermoplastic or other type of material that is not reactive with the ingredients of the developer composition. Preferably the bottle 3 is plastic and has a screw cap 4 which is easily removed and secured. The screw cap 4 is also preferably made of plastic.

The oxidative composition may be contained in a tube, jar, packette, or other form as depicted in FIGS. 4a-c. No matter what type of container is used to store the oxidative composition, the container must be resistant to the ingredients found within the oxidative composition. For example, if the oxidative composition is found in a tube, the tube will most likely be metal, and, if desired, laminated or coated with materials that make the tube walls impervious to the oxidative composition ingredients. In the case where a jar is used to contain the oxidative composition, it is most preferably made from an inert material such as glass.

The aftercare composition, whether in the form of a shampoo, conditioner, or other type of hair product may be contained in a variety of containers. For example, FIG. 3a depicts the aftercare composition contained in a squeeze tube. FIG. 3b depicts the aftercare composition contained in a packette. FIG. 3c depicts the aftercare composition contained in a bottle with a removable cap. In all cases the materials used to make the containers for the aftercare composition must be impervious to the ingredients found therein. The color of the aftercare composition is specifically selected to complement the color of the hair that is obtained when the hair is treated with the mixture of the oxidative composition and developer composition. Typically, the best color matches are obtained when the hair is oxidatively colored in one level, and the aftercare composition colors the hair in a tonality that is similar to the tone of the oxidatively colored hair.

The consumer purchases the kit in a retail store, for example. The hair color process is initiated by combining the oxidative composition with the developer composition by adding the oxidative composition to the bottle 3 containing the developer composition 2. The combined ingredients are mixed well and applied to the hair. Typically, the kit will also contain a nozzle 5 or other type of application device. The consumer removes the cap 4 from the container 3 after the oxidative composition has been added to the developer composition and mixed well. The nozzle 5 is secured to the container 3 and the mixture within is applied to the hair. The hair is colored for the desired period of time. After the desired period of time has elapsed, the consumer removes the hair color mixture from the hair by rinsing well with water. The aftercare composition may then be applied directly after completion of the coloring procedure, and at regular intervals until the next oxidative coloring procedure as will be further described in the method section herein. In the most preferred kit of the invention, the oxidative composition and the aftercare composition are color matched, meaning that if the oxidative composition colors the hair in any particular shade level (as set forth above), the aftercare composition will provide tone to the oxidatively colored hair. In some cases the tone provided by the oxidative color is in the same general color level as the oxidatively colored hair, or one or two levels above or below it. In some other cases, the aftercare composition will provide a tone that is completely different from the oxidatively colored hair, for example, when an individual with oxidatively colored blonde hair applies an aftercare composition have red tones. For example, if the consumer oxidatively colors her hair in a Level 4 shade, if the consumer desires to maintain the same general hair shade, the aftercare composition in the kit will contain semi-permanent dyes that will provide the same general color tone to the hair after application thereof. On the other hand, if the consumer desires to have hair with a unique visual effect, it is possible that the aftercare composition will provide considerably different tones to the hair.

II. The Method

The invention also comprises a method for improving color deposition and fade resistance of oxidatively colored hair by applying an aftercare composition containing at least one semi-permanent dye immediately after oxidatively coloring the hair, and repeating such applications of aftercare composition at intermittent time periods between oxidative dye sessions.

In particular, the hair is colored by combining the oxidative composition and the developer composition and mixing the two components well, then applying them to the hair for the desired period of time to color the hair. Generally, about 1 to 2 parts of oxidative composition and about 1 to 2 parts of developer composition are combined to form an oxidative composition that will color the hair. The most desired mixture is obtained by combining about 1.5 parts developer composition and 1 part oxidative composition to form an oxidative dye mixture. In general, this mixture may be applied to hair for time periods ranging from about 5 to 60 minutes to achieve the desired hair color. The mixture is then rinsed well from the hair with water. Then, the aftercare composition is applied to the hair and allowed to remain on the hair for time periods ranging from fractions of a second to about 20 minutes, preferably 5 second to about 5 minues, more preferably 30 seconds to about 2 minutes, including all numbers in between such ranges. The aftercare composition is then rinsed from the hair well with water.

Then, the hair may be further treated with the aftercare composition intermittently until the next oxidative procedure. The after care composition may be applied to the hair every 1 to 30 days by substituting the aftercare composition for the hair conditioner or shampoo that is usually used by the consumer, with reapplication every 7 to 10 days being optimum. The use of the aftercare composition after oxidative coloring of the hair will result in maintaining the color of the hair and reducing the tendency of the hair color to fade and wash out.

III. The Compositions in the Kit and Method

A. Oxidative Composition

The oxidative composition is an aqueous based composition generally comprising from about 0.01 -95%, preferably about 0.05-95%, preferably about 0.1-85% by weight of the total composition of water. The oxidative composition may be in the form of a solution or emulsion. If the latter, the emulsion generally comprises from about 0.01-95%, preferably abtou 0.05-85%, more preferably about 0.1-80% by weight of the total composition of water and about 0.01 -80%, preferably about 0.1 -65%, preferably about 0.5-50% by weight of the total composition of an oily phase. The oxidative composition may comprise a variety of other ingredients as further described herein.

1. Primary Intermediates.

The oxidative composition preferably comprises at least one primary intermediate and, optionally, at least one coupler for the formation of oxidative dyes. If present, suggested ranges of primary intermediates are about 0.0001 -6%, preferably about 0.0005-5.5%, more preferably about 0.001-5% by weight of the total oxidative composition. Such primary intermediates are well known for use in hair color, and include ortho or para substituted aminophenols or phenylenediamines, including para-phenylenediamines of the formula:

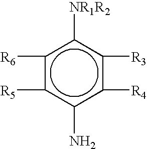

wherein R₁ and R₂ are each independently hydrogen, C₁₋₆ alkyl, or C₁₋₆ alkyl substituted with one or more hydroxy, methoxy, methylsulphonylamino, aminocarbonyl, furfuryl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₆ alkyl substituted with one or more hydroxy or amino groups.

Specific examples of suitable primary intermediates include para-phenylenediamine, 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, and derivatives thereof, and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.

2. Color Coupler

The oxidative composition optionally comprises from about 0.0001-10%, more preferably about 0.0005-8%, most preferably about 0.001-7% by weight of the total oxidative composition of one or more color couplers. Suitable color couplers include, for example, those having the general formula:

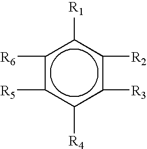

wherein R₁ is unsubstituted hydroxy or amino, or hydroxy or amino substituted with one or more C₁₋₆ hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄ together may form a methylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methyl pyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy- 1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamionphenoxyethanol, and mixtures thereof.

Preferred couplers include resorcinol, 1-naphthol, 2-methylresorcinol, 4-amino-2-hydroxy toluene, m-aminophenol, 2,4-diaminophenoxyethanol, phenyl methyl pyrazolone, their salts, or mixtures.

In the haircolor industry, haircolor is classified into one of ten levels as follows:

```
  1 = very black
  2 = bright black
  3 = very dark brown
  4 = dark brown
  5 = medium brown
  6 = light brown
  7 = dark blonde
  8 = medium blonde
  9 = light blonde
10 = high lift blonde
```

Set forth in the table below is a non-limiting example of the primary intermediates and the color couplers that may be used in various shades of hair color. Other primary intermediates and couplers may be used in addition to, or in lieu of, those set forth in the Table and nothing herein shall be construed to limit the invention to only those primary intermediates and couplers set forth.

| Primary Intermediates | Couplers | Primary Intermediates | Couplers |
|---|---|---|---|
| Level 1 - Very Black | | Level 2 - Bright Black | |
| p-phenylenediamine | m-aminophenol | p-phenylenediamine | resorcinol |
| p-phenylenediamine | resorcinol | 2-chloro-P-phenylenediamine | |
| sulfate | | sulfate | |
| 2-chloro-phenylenediamine | 4-amino-2-hydroxytoluene | o-aminophenol | |
| sulfate | | | |
| p-aminophenol | 4-chlororesorcinol | | |
| o-aminophenol | m-aminophenol HCL | | |
| | 2,4-diaminophenoxy | | |
| | ethanol | | |
| | m-phenylenediamine | | |
| | sulfate | | |
| Level 3 - Very Dark Brown | | Level 4 - Dark Brown | |
| p-phenylenediamine | resorcinol | p-phenylenediamine | resorcinol |
| N,N-bis(2-hydroxyethyl)- | 1-naphthol | N,N-bis(2- | 1-naphthol |
| P-phenylenediamine | | hydroxyethyl)-P- | |
| sulfate | | phenylene diamine | |
| | m-aminophenol | sulfate | |
| | | p-aminophenol | m-aminophenol |
| | | | phenyl methyl |
| | | | pyrazolone |
| | | o-aminophenol | 4-amino-2- |
| | | | hydroxytoluene |
| Level 5 - Medium Brown | | Level 6 - Light Brown | |
| p-phenylenediamine | resorcinol | p-phenylenediamine | resorcinol |
| N,N-bis(2-hydroxyethyl)- | 1-naphthol | N,N-bis(2-hydroxyethyl)- | 1-naphthol |
| P-phenylenediamine | | P-phenylenediamine | |
| sulfate | | sulfate | |
| p-aminophenol | m-aminophenol | p-aminophenol | m-aminophenol |
| o-aminophenol | phenyl methyl | | phenyl methyl |
| | pyrazolone | | pyrazolone |
| | 2-methylresorcinol | | 4-amino-2-hydroxytoluene |
| | 4-amino-2-hydroxtoluene | | 2-methylresorcinol |
| Level 7 - Dark Blonde | | Level 8 - Medium Blonde | |
| p-phenylenediamine | resorcinol | p-phenylenediamine | resorcinol |
| N,N-bis(2-hydroxyethyl)- | 1-naphthol | N,N-bis(2- | 1-naphthol |
| P-phenylenediamine | | hydroxyethyl)-P- | |
| sulfate | | phenylenediamine | |
| | | sulfate | |
| p-aminophenol | phenyl methyl | p-aminophenol | m-aminophenol |
| | pyrazolone | | |
| o-aminophenol | | | phenyl methyl |
| | | | pyrazolone |
| | | | 4-amino-2- |
| | | | hydroxytoluene |
| Level 9 - Light Blonde | | Level 10 - High Lift Blonde | |
| p-phenylenediamine | resorcinol | p-phenylenediamine | resorcinol |
| N,N-bis(2- | 4-amino-2-hydroxytoluene | N,N-bis(2-hydroxyethyl)- | 1-naphthol |
| hydroxyethyl)-P- | | P-phenylenediamine | |
| phenylenediamine | | sulfate | |
| sulfate | | | |
| p-aminophenol | phenyl methyl | | phenyl methyl |
| | pyrazolone | | pyrazolone |
| o-aminophenol | 2-methylresorcinol | | 2-methylresorcinol |
| | 1-naphthol | | |

3. Alkalizing Agent

The oxidative composition preferably contains one or more alkalizing agents in a range of about 0.0001-15%, preferably about 0.005-10%, more preferably about 0.01-5% based on the total weight of the oxidative composition. The term “alkalizing agent” means an ingredient that is capable of imparting alkalinity (e.g. a pH of greater than 7) to the oxidative composition. Suitable alkalizing agents include ammonium hydroxide, metal hydroxides, alkanolamines, sodium silicate, metal carbonates, sodium metasilicate, and mixtures thereof. Suitable metal hydroxides and carbonates include alkali metal and alkaline earth metal hydroxides or carbonates. Examples of such metal hydroxides include sodium, potassium, lithium, calcium, magnesium and so on. A particularly preferred alkaline earth metal hydroxide is sodium hydroxide. Suitable alkanolamines include mono-, di-, and trialkanolamines such as monoethanolamine (MEA), diethanolamine (DEA), triethanolamine (TEA), 2-aminobutanol, aminoethyl propanediol, aminomethyl propanediol, bis-hydroxyethyl tromethamine, diethanolamine, diethyl ethanolamine, diisopropanolamine, dimethylamino methylpropanol, dimethyl MEA, isopropanolamine, methylethanolamine, mixed isopropanolamines, triisopropanolamine, tromethamine, and mixtures thereof. A particularly preferred alkanolamine is MEA.

The alkalizing agent present in the oxidative composition may react with other ingredients in the mixture in situ, such as fatty acids, proteins or hydrolyzed proteins, and the like. Depending on the amount of alkalizing agent present and the presence or absence of ingredients that will react with the alkalizing agent, it is possible that the alkalizing agent may be completely reacted in situ, partially reacted in situ, or not reacted at all if there are no other ingredients in the composition that will react with the alkalizing agent.

Most preferred are dye mixtures that contain ammonium hydroxide in combination with a second alkalizing agent such as an alkanolamine. In general, the amount of alkalizing agent found in the dye mixture will depend on the color of the dye. Less alkalizing agent is used with darker hair colors in Levels 1-6, whereas more alkalizing agent is necessary in lighter shades having Levels 7-10.

In the most preferred embodiment of the invention the oxidative composition contains ammonium hydroxide in addition to a second alkalizing agent selected from sodium hydroxide, alkanolamine, or metal hydroxide. Preferably the second alkalizing agent is an alkanolamine.

4. Fatty Acids

The oxidative composition may contain one or more fatty acids, and if so suggested ranges are about 0.001-15%, preferably 0.005-10%, most preferably 0.01-8% by weight of the total composition. If fatty acids are present they will react with the alkalizing agent to form soap in situ, which provides a more shampoo-like character to the oxidative composition once it is applied to hair. Such fatty acids are of the general formula RCOOH wherein R is a straight or branched chain, saturated or unsaturated C₆₋₃₀ alkyl. Examples of suitable fatty acids include oleic acid, stearic acid, myristic acid, linoleic acid, and so on. Particularly preferred is oleic acid.

5. Conditioners

Preferably the oxidative composition comprises one or more conditioners that exert a conditioning effect on hair. A variety of conditioners are suitable including cationic polymers, oily conditioning agents, fatty alcohols, proteins, and so on. A combined total weight of conditioners ranges from about 0.01-25%, preferably 0.05-20%, more preferably 1-15% by weight of the total oxidative composition.

(a) Cationic Polymers

A variety of cationic polymers are suitable such as quaternary derivatives of cellulose ethers or guar derivatives, copolymers of vinylpyrrolidone, polymers of dimethyldiallyl ammonium chloride, acrylic or methacrylic polymers, quaternary ammonium polymers, and the like.

(i) Quaternary Derivatives of Cellulose

Examples of quaternary derivatives of cellulose ethers are polymers sold under the tradename JR-125, JR-400, JR-30M. Suitable guar derivatives include guar hydroxypropyl trimonium chloride.

(ii) Copolymers of Vinylpyrrolidone

Copolymers of vinylpyrrolidone having monomer units of the formula:

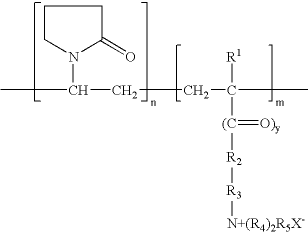

wherein R¹ is hydrogen or methyl, preferably methyl;

y is 0 or 1, preferably 1

R² is 0 or NH, preferably NH;

R³ is CₓH₂ₓ where x is 2 to 18, or —CH₂—CHOH—CH₂ , preferably CₓH₂ₓ where x is 2;

R⁴ is methyl, ethyl, phenyl, or C₁₋₄ substituted phenyl, preferably methyl; and

R⁵ is methyl or ethyl, preferably methyl.

(iii) Polymers of Dimethyldiallylammonium Chloride

Homopolymers of dimethyldiallylammonium chloride, or copolymers of dimethyldiallylammonium chloride and acrylamide are also suitable. Such compounds are sold under the tradename MERQUAT by Calgon.

(iv) Acrylic or Methacrylic Acid Polymers

Homopolymers or copolymers derived from acrylic or methacrylic acid, selected from monomer units acrylamide, methylacrylamide, diacetone-acrylamide, acrylamide or methacrylamide substituted on the nitrogen by lower alkyl, alkyl esters of acrylic acid and methacrylic acid, vinylpyrrolidone, or vinyl esters are suitable for use.

(v) Polymeric Quaternary Ammonium Salts

Also suitable are polymeric quaternary ammonium polymers such as Polyquatemium 10, 28 31,33,34, 35, 36, 37, and 39.

(vi) Diquaternary Polydimethylsiloxanes

Also suitable are diquaternary polydimethylsiloxanes such as Quaternium-80, sold by Goldschmidt Corporation under the tradename ABIL-Quat 3272.

Examples of other cationic polymers that can be used in the compositions of the invention are disclosed in U.S. Pat. Nos. 5,240,450 and 5,573,709, which are hereby incorporated by reference.

Particularly preferred are conditioners Polyquatemium 10 and Polyquaternium 28. Polyquatemium-10 is the polymeric quaternary ammonium salt of hydroxyethyl cellulose reacted with trimethyl ammonium substituted epoxide. Polyquatemium-28 is the polymeric quaternary ammonium salt consisting of vinyl pyrrolidone and dimethylaminopropyl methacrylamide monomers.

(b) Oily Conditioning Agents

Also suitable are a variety of oily materials that provide good conditioning effect to hair. Suitable oils are liquid at room temperature and may comprise esters, hydrocarbons, and the like. Preferably the composition comprises 0.001-20%, more preferably 0.005-15%, most preferably 0.01-10% by weight of the total oxidative composition. Particularly preferred oily conditioning agents are oils extracted from vegetable sources, such as meadowfoam seed oil.

(c) Nonionic Silicones

Also suitable as conditioning agents are one or more silicones. Suitable silicone hair conditioning agents include volatile or nonvolatile nonionic silicone fluids, silicone resins, and silicone semi-solids or solids.

Volatile silicones are linear or cyclic silicones having a measureable vapor pressure, which is defined as a vapor pressure of at least about 2 mm. of mercury at 20° C. Examples of volatile silicones are cyclic silicones having the general formula:

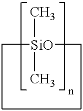

where n=3-7.

Also, linear volatile silicones that may be used in the compositions of the invention have the general formula:

(CH₃)₃Si—O—[Si(CH₃)₂—O]ₙ—Si(CH₃)₃

where n=0-7, preferably 0-5.

The silicone hair conditioning agent may comprise water insoluble nonvolatile silicone fluids including polyalkyl siloxanes, polyaryl siloxanes, polyalkylaryl siloxanes, polyether siloxane copolymers, amine-functional silicones, and mixtures thereof. Such silicones have the following general formula:

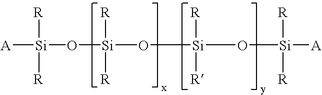

wherein R and R′ are each independently alkyl, aryl, or an alkyl substituted with one or more amino groups, and x and y are each independently 0-100,000, with the proviso that x+y equals at least one and A is siloxy endcap unit. Preferred is where A is methyl, R is methyl, and R′ is an alkyl substituted with at least two amino groups, most preferably an amine-functional silicone having the formula:

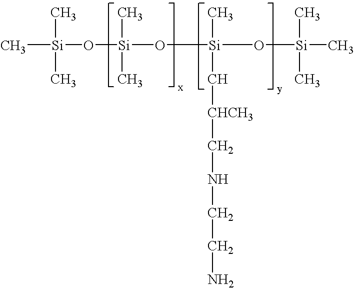

which is known by the CTFA name trimethylsilylamodimethicone.

The silicone hair conditioning agent may also be a silicone polymer having the following general formula:

[(RR′R″)₃SiO_{1/2}]ₓ[SiO₂]_{y}

wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{1/2} units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Coming Corporation under the tradename 2-0749 and 2-0747, each of which is a blend of about 40-60% volatile silicone and 40-60% trimethylsiloxy silicate. Dow Coming 2-0749, in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

6. Surfactants or Emulsifiers

The oxidative composition preferably comprises one or more surfactants that assist in maintaining the composition in the preferred emulsion form and aid in the foaming capability of the composition. Suitable surfactants include anionic surfactants, nonionic surfactants, amphoteric surfactants, and the like.

(a) Nonionic Surfactants

Suggested ranges of nonionic surfactant are about 0.01-10%, preferably about 0.05-8%, more preferably about 0.1-7% by weight of the total oxidative composition. Suitable nonionic surfactants include alkoxylated alcohols or ethers, alkoxylated carboxylic acids, sorbitan derivatives, and the like.

Suitable alkoxylated alcohols, or ethers, are formed by the reaction of an alcohol with an alkylene oxide, usually ethylene or propylene oxide. Preferably the alcohol is a fatty alcohol having 6 to 30 carbon atoms, and a straight or branched, saturated or unsaturated carbon chain. Examples of such ingredients include steareth 2-30, which is formed by the reaction of stearyl alcohol and ethylene oxide where the number of repeating ethylene oxide units is 2 to 30; Oleth 2-30 which is formed by the reaction of oleyl alcohol and ethylene oxide where the number of repeating ethylene oxide units is 2 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Particularly preferred are Steareth-2 1, which is the reaction product of a mixture of stearyl alcohol with ethylene oxide, and the number of repeating ethylene oxide units in the molecule is 21, and Oleth-20 which is the reaction product of oleyl alcohol and ethylene oxide wherein the number of repeating ethylene oxide units in the molecule is 20.

Also suitable as the nonionic surfactant are alkyoxylated carboxylic acids, which are formed by the reaction of a carboxylic acid with an alkylene oxide or with a polymeric ether. The resulting products have the general formula:

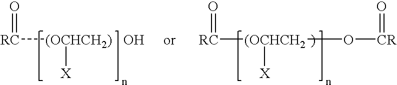

where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable are various types of alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

(b) Anionic Surfactants

If desired the dye composition may contain one or more anionic surfactants. Together with the soap formed by the reaction of the fatty acid and alkanolamine or metal hydroxide, the ingredients provide the composition with the characteristics of shampoo. Preferred ranges of anionic surfactant are about 0.1-25%, preferably 0.5-20%, more preferably 1-15% by weight of the total oxidative composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:

R₁—SO₃-M

wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and 20 tallow based soaps. Preferred salts are ammonium, potassium, and sodium salts.

Still another class of anionic surfactants include N-acyl amino acid surfactants and salts thereof (alkali, alkaline earth, and ammonium salts) having the formula:

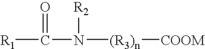

wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

Also suitable are amphoteric and zwitterionic surfactants. Examples of amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

7. Thickening Agents

Preferably the oxidative composition contains one or more thickening agents that increase the viscosity of the composition such that when it is applied to hair it doesn't run. The amount of thickening agent if present is about 0.001-5%, preferably about 0.005-4%, more preferably about 0.005-3% by weight of the total oxidative composition.

A variety of thickening agents are suitable including low melting point waxes, carboxyvinyl polymers, and the like. Particularly preferred thickening agents are low melting point waxes such as emulsifying wax, fatty alcohols (e.g. stearyl alcohol, cetearyl alcohol, behenyl alcohol, and the like). Preferred are cetearyl alcohol and emulsifying wax.

8. Solvents

It may be desirable to include one or more solvents in the dye composition. Such solvents assist in solubilizing the primary intermediate dyestuff and coupler dyestuff components, in addition to the other ingredients in the composition. The solvent is preferably present at about 0.01-10%, preferably 0.05-8%, more preferably 0. 1-7% by weight of the total oxidative composition. Suitable solvents include C₂₋₄ alkanols such as ethanol, isopropanol, propanol, etc., as well as askoxydiglycols such as ethoxydiglycol. The preferred solvent comprises ethoxydiglycol.

9. Chelating Agents

Preferably, the oxidative composition contains one or more chelating agents that are capable of chelating the metal ions found in water. If water contains too many extraneous metal ions they can interfere with the coloration process. Preferred ranges of chelating agent are 0.001-5%, preferably 0.005-4%, more preferably 0.01-3% by weight of the total composition. Preferred chelating agents are EDTA, HEDTA, and sodium or potassium salts thereof.

10. Antioxidants

The oxidative composition may also contain one or more antioxidants as described herein with respect to the dye composition and in the same ranges by weight.

Various other ingredients such as preservatives may also be incorporated into the claimed compositions.

In the most preferred embodiment of the invention the oxidative compositions are as described in U.S. patent application Ser. No.09/852,982, filed May 10, 2001, entitled Methods and Compositions for Coloring Hair, which is hereby incorporated by reference in its entirety.

B. The Developer Composition

The developer composition (also referred to as an activator or peroxide composition) is, in its simplest form, is an aqueous solution of a peroxide oxidizing agent, preferably hydrogen peroxide, but other organic or inorganic peroxide oxidizing agents are also suitable. Preferably the developer composition comprises 1-99%, preferably 10-99%, more preferably 60-97% of water, and about 5-20%, preferably 6-15%, more preferably 7-10% by weight of the total developer composition of the peroxide oxidizing agent. Aqueous hydrogen peroxide compositions are generally sold in the form of 10, 20, 25, and 30 volume hydrogen peroxide. The 25 volume hydrogen peroxide developer composition contains about 7.5% by weight of the total composition of hydrogen peroxide. The 30 volume hydrogen peroxide developer composition contains about 9% by weight of the total composition of hydrogen peroxide. If desired, the developer composition may contain a variety of other ingredients that enhance the aesthetic properties and contribute to more efficient coloring of hair. Preferred developer compositions comprise about:

0.5-25% hydrogen peroxide,

0.1-10% of a conditioner,

0.01-5% of a thickener, and

1-99% water.

1. Conditioners

The developer composition may contain one or more conditioners that exert a conditioning effect on hair. The conditioners mentioned above with respect to the oxidative compositions are also suitable for use in the developer composition, and in the same suggested ranges. Also suitable are various types of cationic silicones as further described below.

(a) Cationic Silicones

As used herein, the term “cationic silicone” means any silicone polymer or oligomer having a silicon backbone, including polysiloxanes, having a positive charge on the silicone structure itself Cationic silicones that may be used in the compositions of the invention include those corresponding to the following formula, where the ratio of D to T units, if present, are greater than about 80 D units to 1 T unit:

(R)ₐG₃₋ₐ-Si(OSiG₂)ₙ(OSiG_{b}(R₁)_{2-6b})ₘ—O—SiG₃₋ₐ(R₁)ₐ

in which G is selected from the group consisting of H, phenyl, OH, C₁₋₁₀ alkyl, and is preferably CH₃; and a is 0 or an integer from 1 to 3, and is preferably 0; b is 0 or 1, preferably 1; the sum n+m is a number from 1 to 2,000 and is preferably 50 to 150; n is a number from 0 to 2000, and is preferably 50 to 150; and m is an integer from 1 to 2000, and is preferably 1 to 10; R is a C₁₋₁₀ alkyl, and R₁ is a monovalent radical of the formula C_{q}H_{2q}L in which q is an integer from 2 to 8 and L is selected from the groups:

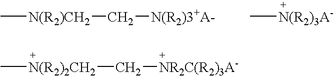

in which R₂ is selected from the group consisting of H, phenyl, benzyl, a saturated hydrocarbon radical, and is preferably an alkyl radical containing 1-20 carbon atoms; and A- is a halide, methylsulfate, or tosylate ion.

2. Thickening Agents

The developer composition may contain one or more thickeners that assist in maintaining an increased viscosity of the final composition resulting from mixture of the oxidative composition and the developer composition. This ensures that the mixture is of a sufficient viscosity to prevent it from dripping or running off the hair onto the user's face or the surrounding environment. Suitable thickeners are those set forth above with respect to the oxidative composition, and in the same ranges. Also suitable are a variety of water soluble anionic thickening polymers such as those disclosed in U.S. Pat. No. 4,240,450, which is hereby incorporated by reference. Suggested ranges of such polymers are about 0.01-5%, preferably 0.05-4%, more preferably 0. 1-3% by weight of the total developer composition. Examples of such anionic polymers are copolymers of vinyl acetate and crotonic acid, graft copolymers of vinyl esters or acrylic or methacrylic acid esters, cross-linked graft copolymers resulting from the polymerization of at least one monomer of the ionic type, at least one monomer of the nonionic type, polyethylene glycol, and a crosslinking agent, and the like. Preferred are acrylate copolymers such as steareth-10 allyl ether acrylate copolymer.

3. Nonionic Surfactants

The developer composition may contain one or more nonionic surfactants which assist in maintaining the composition in stable emulsion form. Suitable nonionic surfactants are the same as those mentioned in above with respect to the oxidative composition, and in the same amounts.

4. Chelating Agents

The developer composition may contain one or more chelating agents as described herein with respect to the oxidative composition, and in the same ranges by weight.

C. The Aftercare Composition

The aftercare composition may be in the form of a shampoo, conditioner, or any other type of hair treatment product. It may be in the form of a mousse, cream, gel, or viscous liquid. Preferably the aftercare composition is in the form of a shampoo or conditioner. In whatever form the aftercare composition is in, the composition comprises at least one water soluble or water dispersible dye that is operable to color hair when it comes into contact therewith, without the addition of any developer composition. The phrase “operable to color hair when contacted therewith” means that the dye alone, without combining with any additional developers or accelerators, will color the hair (as opposed to certain types of oxidative dyes that must be combined with an developer in order to impart color to the hair fiber).

A variety of dyes are suitable including direct dyes, disperse dyes, acid dyes, basic, dyes, direct, dyes, and so on. Suitable amounts of dye preferably range from about 0.0001-20%, preferably about 0.005-15%, more preferably about 0.010-10% by weight of the total composition. Preferred are the compounds that fall into the general category of semi-permanent dyes. Examples of such dyes are set forth below:

Basic Dyes

Suitable basic, or cationic dyes include blues, browns, greens, oranges, reds, and yellows. Suitable blues include Basic Blue 3, 6, 7, 9, 26, 41, 47, and 99. Suitable browns include Basic Browns 4, 16, and 17. Suitable greens include Basic Green 1 and 4. Suitable oranges include Basic Orange 1 and 2. Suitable Reds include Basic Red 1, 2, 22, 46, 76, and 118. Suitable violets include Basic Violet 1, 3, 4, 10, 11:1, 14, and 16. Suitable yellows include Basic Yellow 11, 28, and 57.

Suitable basic dyes for use in the claimed compositions are set forth in the CTFA Cosmetic Ingredient Handbook, Eighth Edition, pages 117-124, which are hereby incorporated by reference in their entirety.

HC Dyes

Also suitable for use in the compositions are various HC dyes such as blue, brown, green, orange, red, violet, and yellow. Suitable blues include HC Blue 2, 4, 5, 6, 7, 8, 9, 10, 11, 12, 13, and 14. Suitable browns include HC Brown I and 2. Suitable greens include HC Green 1. Suitable oranges include HC Orange 1, 2, 3, and 5. Suitable reds include HC Red 1, 3, 7, 8, 9, 10, 11, 13, and 14. Suitable violets include HC Violet 1 and 2. Suitable yellows include HC Yellow 2, 4, 5, 6, 7, 8, 9, 10, 11, 13, 14, and 15. Such HC dyes are set forth on pages 615-623 of the CTFA Cosmetic Ingredient Handbook, Eighth Edition, 2000, which is hereby incorporated by reference in its entirety.

Acid Dyes

Also suitable for use in the compositions are various acid dyes such as black, blue, brown, green, orange, red, violet, and yellow. Examples of Acid Black are numbers 1 and 52. Suitable blues include Acid Blue 1, 3, 9, 62, and 74, including Lakes thereof. Examples of browns and greens include Acid Brown 13 and Acid Green 1, 25, and 50, respectively. Suitable oranges include Acid Orange 3, 6, 7, and 24. Suitable reds include Acid Red 14, 18, 27, 33, 35, 51, 52, 73, 87, 92, 95, 184, and 195. Suitable violets include Acid Violet 9 and 43. Suitable yellows include Acid Yellow 1, 3, 23, and 73. In each case the dyes may be Lakes thereof. Such Acid dyes are set forth on pages 13-23 of the CTFA Cosmetic Ingredient Handbook, Eighth Edition, 2000, which is hereby incorporated by reference in its entirety.

Direct and Disperse Dyes

Also suitable are various types of dyes referred to as direct dyes or disperse dyes. Suitable direct dyes include Direct Black 51, Direct Blue 86, Direct Red 23, 80, and 81; Direct Violet 48, and Direct Yellow 12. Such direct dyes are set forth on pages 469-471 of the CTFA Cosmetic Ingredient Handbook, Eighth Edition, 2000, which is incorporated by reference in its entirety.

Suitable disperse dyes include Disperse Black 9, Disperse Blue 1, 3, and 7; Disperse Brown 1, Disperse Orange 3, Disperse Red 11, 15, and 17; and Disperse Violet 1, 4, and 15. Such disperse dyes are as set forth on 491-493 of the CTFA Cosmetic Ingredient Handbook, Eighth Edition, 2000, which is hereby incorporated by reference in its entirety.

For example, oxidatively colored hair in levels 1 through 10 may be treated with aftercare compositions that will provide the tones set forth. In the chart set forth below, the oxidative color of the hair is specified according to level in the left hand column and the various tones that may be found with that color in the headings “Neutral”, “Cool”, “Golden” or “Warm”. The semi-permanent dyes that may be incorporated into the aftercare composition to complement the oxidative color tones are set forth in the columns beneath each tone.

| | Oxidative Color Tones | | | |
|---|---|---|---|---|
| Color | Natural/Neutral | Cool | Golden | Warm |
| Blonde | HC Yellow 2 | HC Yellow 2 | HC Yellow 2 | HC Yellow 2 |
| (levels 7, 8, 9, 10) | HC Blue 2 | HC Blue 2 CP | HC Blue 2 CP | HC Blue 2 CP |
| | Disperse Violet 1 | Disperse Violet 1 | HC Yellow 4 | Disperse Violet 1 |
| | Acid Orange 3 | Acid Orange 3 | Acid Orange 3 | HC Red 3 |
| | Basic Yellow 57 | Basic Yellow 57 | Basic Yellow 57 | Disperse Orange 3 |
| | Basic Red 76 | Basic Brown 16 | Basic Blue 99 | HC Yellow 4 |
| | Basic Blue 99 | Basic Blue 99 | Basic Red 76 | Basic Yellow 57 |
| | | | | Basic Red 76 |
| Brown (levels 2-10) | HC Yellow 2 | HC Yellow 2 | HC Yellow 2 | HC Yellow 2 |
| | HC Yellow 4 | HC Yellow 4 | HC Yellow 4 | HC Yellow 4 |
| | HC Red 3 | HC Red 3 | HC Red 3 | HC Red 3 |
| | HC Red 13 | HC Red 13 | HC Red 13 | HC Red 13 |
| | HC Blue 2 CP | HC Blue 2 CP | HC Blue 2 CP | HC Blue 2 CP |
| | Basic Brown 16 | HC Violet 1, 4 | Basic Brown 17 | Disperse Orange 3 |
| | Basic Brown 17 | bis | Basic Yellow 57 | Basic Brown 16 |
| | Basic Blue 99 | Basic Blue 99 | Basic Red 76 | Basic Red 76 |
| | | Basic Brown 17 | | Basic Brown 17 |
| | | Basic Brown 16 | | |
| Red (level 2-10) | — | HC Yellow 2 | HC Yellow 2 | HC Yellow 2 |
| | | HC Yellow 4 | HC Yellow 4 | HC Yellow 4 |
| | | HC Red 3 | HC Red 3 | HC Red 3 |
| | | Disperse Orange 3 | Disperse Orange 3 | Disperse Orange 3 |
| | | HC Blue 2 CP | Basic Yellow 47 | Basic Red 76 |
| | | | Basic Red 76 | Basic Brown 17 |
| | | | Basic Blue 99 | Basic Blue 99 |
| | | | Basic Brown 16 | |
| Black (level 1-3) | HC Yellow 2 | HC Yellow 2 | | HC Yellow 2 |
| | HC Yellow 4 | HC Yellow 4 | | HC Yellow 4 |
| | HC Red 3 | HC Red 3 | | HC Red 4 |
| | HC Red 13 | HC Red 13 | | HC Red 13 |
| | HC Blue 2 CP | HC Blue 2 CP | | HC Blue 2 CP |
| | HC Violet 1,4- | HC Violet 1,4- | | HC Violet 1,4- |
| | bis | bis | | bis |
| | Basic Blue 99 | Basic Blue 99 | | Basic Blue 99 |
| | Basic Brown 16 | Basic Brown 16 | | Basic Brown 16 |
| | Basic Brown 17 | Basic Brown 17 | | Basic Brown 17 |
| | Basic Yellow 57 | Basic Yellow 57 | | Basic Red 76 |
| | Basic Red 76 | | | Basic Yellow 57 |

The above chart exemplifies some of the types of combinations that may be used to provide desired effects, but is not to be construed as limiting the semi-permanent dyes or the color tones exemplified. Rather, a wide variety of other combinations of oxidative dye colors and tones may be combined with the various types of semi-permanent dyes set forth above, or others not set forth above or herein and the tones may differ also.

1. Aftercare Conditioner

In the case where the after care composition is in the form of a conditioner, the composition may additionally comprise a variety of other ingredients including water, oil, surfactants, emulsifiers, and conditioning agents. Generally hair conditioners are in the form of emulsion comprising from about 0.01-95%, preferably about 0.05-90%, preferably about 0.05-80% by weight of the total composition. Preferably, the aftercare conditioner composition is in the acidic pH range, for example ranging from about 3-7, preferably 5-6. The aftercare conditioner may also comprise the following ingredients:

(a). Conditioners

In addition to the hair conditioners and conditioning ingredients and polymers set forth above with respect to the oxidative composition and the developer composition, and in the same percentage ranges, the aftercare conditioner may also contain one or more cationic quaternary ammonium compounds as conditioning agents. If so, ranges of about 0.001-5%, preferably about 0.002-4%, more preferably about 0.01-3% by weight of the total composition is suggested. Suitable cationic quaternary ammonium compounds include:

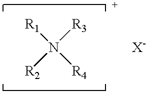

wherein R₁ is an aliphatic group of 1 to 22 carbon atoms, or aromatic, aryl, or alkaryl group having 12 to 22 carbon atoms; R₂ and R₃ are each independently an aliphatic group having 1-22 carbon atoms; and R4 is an alkyl group of from 1 to 3 carbon atoms, and X is an anion selected from halogen, acetate, phosphate, nitrate and methyl sulfate radicals. The aliphatic groups may contain, in addition to carbon atoms, ether linkages as well as amido groups. Suitable quaternary ammonium compounds may be mono-long chain alkyl, di-long chain alkyl, tri-long chain alkyl, and the like. Examples of such quaternary ammonium salts include behenalkonium chloride, behentrimonium chloride, behentrimonium methosulfate, benzalkonium chloride, benzethonium chloride, benzyl triethyl ammonium chloride, cetalkonium chloride, cetrimonium chloride, cetrimonium bromide, cetrimonium methosulfate, cetrimonium tosylate, cetylpyridinium chloride, dibehenyl/diarachidyl dimonium chloride, dibehenyldimonium chloride, dibehenyldimonium methosulfate, dicapryl/dicaprylyl dimonium chloride,

Other quaternary ammonium salts useful as the conditioning agent are compounds of the general formula:

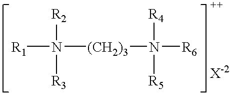

wherein R₁ is an aliphatic group having 16 to 22 carbon atoms, R₂, R₃, R₄, R₅, and R₆ are the same or different and are selected from alkyls having 1 to 4 carbon atoms and X is an anion as above defined.

Amides, which exhibit the general formulas set forth below, are also suitable conditioning agents:

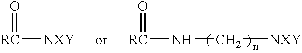

wherein R is a straight or branched chain saturated or unsaturated alkyl having 6 to 30 carbon atoms, n is an integer from 1 to 4, and X and Y are each independently H, or C₁₋₆ lower alkyl. Preferred is an amide of the formula:

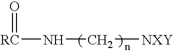

wherein R is a C₁₂₋₂₂ straight or branched chain alkyl, n is an integer from 1 to 4, and X is lower alkyl, preferably methyl.

Also suitable are amidoamine salts, which are the condensation products of fatty acids with a polyfunctional amines, for example, those having the formula RCONH(CH₂)ₙNR₁R₂ where RCO is a fatty acyl group such as stearoyl, R₁ and R₂ are methyl or ethyl, and n is 2 or 3. Examples of such compounds include stearmidopropyl dimethylamine. Particularly preferred are amidoamine compounds complexed with a mild dimer acid, such as di(behenamidopropyl dimethyl amine) dimer dilinoleate or di(linoleamidopropyl dimethyl amine) dimer linoleate. Both ingredients are sold by Alzo, Inc. under the NECON tradename.

Also, quaternary imidazolinium salts having the following general formula are suitable as the cationic conditioning agent:

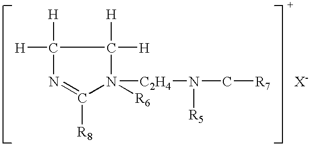

wherein R₅ is hydrogen or a C₁₋₄ alkyl; R₆ is a C₁₋₄ alkyl; R₇ is a C₈₋₂₂ alkyl; and R₈ is hydrogen, or a C₁₋₂₂ alkyl; and X is an anion as defined above.

Also suitable as the cationic hair conditioning agent are salts of fatty primary, secondary, or tertiary amines, wherein the substituted groups have 12 to 22 carbon atoms. Examples of such amines include dimethyl stearamine, dimethyl soyamine, stearylamine, myristylamine, tridecylamine, ethyl stearamine, and so on.

(c). Fatty Alcohols

The aftercare conditioner may also comprise one or more fatty alcohols. If so, suggested ranges include from about 0.01-20%, preferably about 0.05-15%, more preferably about 0.1-10% by weight of the total composition. Such fatty alcohols generally have the formula RCH₂OH wherein R is a straight or branched chain saturated or unsaturated alkyl having at least about 6 to 30 carbon atoms. Examples of fatty alcohols suitable for use include behenyl alcohol, C₉₋₁₅ alcohols, caprylic alcohol, cetearyl alcohol, cetyl alcohol, coconut alcohol, decyl alcohol, lauryl alcohol, cetyl alcohol, myristyl alcohol, oleyl alcohol, palm alcohol, stearyl alcohol, tallow alcohol, and the like. The preferred compositions of the invention include a mixture of cetyl and stearyl alcohols.

(d). Humectants

It may be desirable to include one or more humectants in the aftercare composition. The term “humectant” means an ingredient which has hygroscopic, or water attracting properties. Suitable humectants include di- or polyhydric alcohols such as glycerin, butylene glycol, propylene glycol, glucose, sucrose, and the like. If present, suggested ranges of humectant are from about 0.001-10%, preferably about 0.005-8%, more preferably about 0.01-5% by weight of the total aftercare composition.

(e). Preservatives

Preferred composition contain preservatives and/or chelating agents in amounts ranging from about 0.001-10%, preferably about 0.005-8%, more preferably 0.01-5% by weight of the total composition. Suitable preservatives include methylchloroisothizolinone, methylisothiazolinone, the parabens, and the like. Examples of chelating agents include ethylene diamine tetraacetic acid (EDTA) or salts thereof.

2. Aftercare Shampoo

If desired, the aftercare composition may be in the form of a shampoo. If so, the composition is generally in an aqueous form, and in addition to water will comprise one or more cleansing surfactants. The term “cleansing surfactant” means a surfactant that is traditionally known to provide cleansing and foaming in shampoo compositions. The cleansing surfactant may be present ranging from about 0.01-45%, preferably about 0.05-40%, more preferably about 0. 1-35% by weight of the total composition, and the water present at about 0.01-99%, preferably about 5-95%, more preferably about 7-95% by weight of the total composition. The aftercare shampoo may be either alkaline or acidic in nature depending on the types of dyes being used. For example, if alkaline dyes are used, it is preferred that the aftercare shampoo have an alkaline pH, ranging from greater than 7 to about 11, preferably about 9 to 10 including all numbers in between. On the other hand, if acidic dyes are used, it is preferred that the aftercare shampoo have an acid pH, ranging from about 3 to less than about 7, preferably about 4 to 5, including all numbers in between.

Suitable cleansing surfactants may be anionic, nonionic, amphoteric, or zwitterionic. Examples of such surfactants include:

(a) Anionic Surfactants

Suitable anionic surfactants are those set forth above with respect to the oxidative compositions and in the same general percentage ranges.

(b). Nonionic Surfactants

In addition to the nonionic surfactants set forth above with respect to the oxidative composition, and in the same percentage ranges, the composition can contain one or more of the following nonionic surfactants in lieu of, or in addition to, the anionic surfactant. Nonionic surfactants are generally compounds produced by the condensation of alkylene oxide groups with a hydrophobic compound. Classes of nonionic surfactants include:

(i) Long chain dialkyl sulfoxides containing one short chain alkyl or hydroxy alkyl radical of from about 1 to 3 carbon atoms and one long hydrophobic chain which may be an alkyl, alkenyl, hydroxyalkyl, or ketoalkyl radical containing from about 8 to 20 carbon atoms, from 0 to 10 ethylene oxide moieties, and 0 or 1 glyceryl moiety.

(ii) Polysorbates, such as sucrose esters of fatty acids. Examples of such materials include sucrose cocoate, sucrose behenate, and so on.

(iii) Polyethylene oxide condensates of alkyl phenols, for example the condensation products of alkyl phenols having an alkyl group of 6 to 20 carbon atoms with ethylene oxide being present in amounts of about 10 to 60 moles of ethylene oxide per mole of alkyl phenol.

(iv) Condensation products of ethylene oxide with the reaction product of propylene oxide and ethylene diamine.

(v) Condensation products of aliphatic alcohols having 8 to 18 carbon atoms with ethylene oxide, for example a coconut alcohol/ethylene oxide condensate having 10 to 30 moles of ethylene oxide per mole of coconut alcohol, the coconut alcohol fraction having 10 to 14 carbon atoms.

(vi) Long chain tertiary amine oxides such as those corresponding to the general formula:

R₁R₂R₃NO

wherein R₁ contains an alkyl, alkenyl or monohydroxyalkyl radical ranging from about 8 to 18 carbon atoms in length, from 0 to about 10 ethylene oxide moieties, and from 0 to about 1 glyceryl moiety and R₂ and R₃ are each alkyl or monohydroxyalkyl groups containing from about 1 to about 3 carbon atoms.

(vii) Long chain tertiary phosphine oxides corresponding to the general formula:

RR₁R₂PO

wherein R contains an alkyl, alkenyl, or monohydroxyalkyl radical having 8 to 18 carbon atoms, from 0-10 ethylene oxide moieties and 0 or 1 glyceryl moiety, and R₂ and R₃ are each alkyl or monohydroxyalkyl group containing from about 1 to 3 carbon atoms.

(viii) Alkyl polysaccharides having a hydrophobic group of 6 to 30, preferably 10, carbon atoms and a polysaccharide group such as glucose, galactose, etc. Suitable alkyl polysaccharides are octyl, nonydecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses, and so on.

(ix) Polyethylene glycol (PEG) glyceryl fatty esters, having the formula

RC(O)OCH₂CH(OH)CH₂(OCH₂CH₂)ₙOH

wherein n is 5-200 and RC(O)— is a hydrocarbylcarbonyl group wherein R is preferably an aliphatic radical having 7 to 19 carbon atoms.

(x) Other nonionic surfactants that may be used include C₁₀₋₁₈ alkyl(C₁₋₆)polyhydroxy fatty acid amides such as C₁₂₋₁₈ methylglucamides, N-alkoxy polyhydroxy fatty acid amides, N-propyl through N-hexyl C₁₂₋₁₈ glucamides and so on.

(c). Amphoteric Surfactants

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds having the general formula:

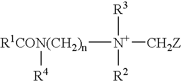

wherein R₁ is C₈₋₂₂ alkyl or alkenyl, preferably C₁₂₋₁₆; R² is hydrogen or CH₂CO₂M, R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium. cation. Examples of such materials are marketed under the tradename MIRANOL, by Miranol, Inc.

Also suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants includ aminoalkanoates of the formula

R—NH(CH₂)ₙCOOM

or iminodialkanoates of the formula:

R—N[(CH₂)ₘCOOM]₂

and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof.

Zwitterionic surfactants are also suitable for use in the compositions of the invention. The general formula for such surfactants is:

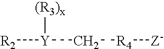

wherein R₂ contains an alkyl, alkenyl or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

Zwitterionics include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)earboxymethyl betaine, stearyl bis-(2-hydroxypropyl)carboxymethyl betaine, oleyl dimethyl gamma-carboxylethyl betaine, and mixtures thereof. Also suitable are sulfo- and amido- betaines such as coco dimethyl sulfopropyl betaine, stearyl dimethyl sulfopropyl betaine, and the like.

The aftercare shampoo may also comprise a variety of other ingredients include conditioning ingredients, humectants, preservatives, botanicals, and other ingredients mentioned above with respect to the conditioner aftercare composition and in the same percentage ranges.

The invention will be further described in connection with the following examples, that are set forth for the purposes of illustration only.

### EXAMPLE 1

Oxidative dye compositions were prepared as follows:

| | | | | | | Ultra | | |
|---|---|---|---|---|---|---|---|---|
| | | Medium | | Light | | Light | | |
| | Dark | Golden | Medium | Cool | Medium | Natural | Dark | Light |
| | Brown | Blonde | Auburn | Brown | Brown | Blonde | Blonde | Auburn |
| Water | QS | QS | QS | QS | QS | QS | QS | QS |
| Erythorbic acid | 0.20 | 0.20 | 0.20 | 0.20 | 0.20 | 0.20 | 0.20 | 0.20 |
| Sodium sulfite | 0.50 | 0.50 | 0.50 | 0.50 | 0.50 | 0.50 | 0.50 | 0.50 |
| Ethoxydiglycol | 5.00 | 5.00 | 5.00 | 5.00 | 5.00 | 5.00 | 5.00 | 5.00 |
| Tetrasodium | 0.80 | 0.80 | 0.80 | 0.80 | 0.80 | 0.80 | 0.80 | 0.80 |
| EDTA | | | | | | | | |
| Ethanolamine | 3.00 | 3.00 | 3.00 | 3.00 | 3.00 | 3.00 | 3.00 | 3.00 |
| Botanical | 0.80 | 0.80 | 0.80 | 0.80 | 0.80 | 0.80 | 0.80 | 0.80 |
| blend* | | | | | | | | |
| Sodium | 0.50 | 0.50 | 0.50 | 0.50 | 0.50 | 0.50 | 0.50 | 0.50 |
| benzotriaolyl | | | | | | | | |
| butylphenol | | | | | | | | |
| sulfonate, | | | | | | | | |
| buteth-3, | | | | | | | | |
| tributyl citrate | | | | | | | | |
| Dye mixture | 2.212 | 0.658 | 2.105 | 0.927 | 1.354 | 0.003 | 1.341 | 1.59 |
| Ammonium | 2.00 | 2.00 | 2.00 | 2.00 | 2.00 | 2.00 | 2.00 | 2.00 |
| lauryl sulfate | | | | | | | | |
| Oleic acid | 12.50 | 12.50 | 12.50 | 12.50 | 12.50 | 12.50 | 12.50 | 12.50 |
| Cetearyl alcohol | 4.00 | 4.00 | 4.00 | 4.00 | 4.00 | 4.00 | 4.00 | 4.00 |
| Emulsifying | 2.00 | 2.00 | 2.00 | 2.00 | 2.00 | 2.00 | 2.00 | 2.00 |
| wax | | | | | | | | |
| Oleth-20 | 1.00 | 1.00 | 1.00 | 1.00 | 1.00 | 1.00 | 1.00 | 1.00 |
| Steareth-21 | 0.70 | 0.70 | 0.70 | 0.70 | 0.70 | 0.70 | 0.70 | 0.70 |
| Meadowfoam | 0.75 | 0.75 | 0.75 | 0.75 | 0.75 | 0.75 | 0.75 | 0.75 |
| Seed Oil | | | | | | | | |
| Oleyl alcohol | 0.40 | 0.40 | 0.40 | 0.40 | 0.40 | 0.40 | 0.40 | 0.40 |
| Polyquaternium- | 0.20 | 0.20 | 0.20 | 0.20 | 0.20 | 0.20 | 0.20 | 0.20 |
| 10 | | | | | | | | |
| Polyquaternium- | 0.50 | 0.50 | 0.50 | 0.50 | 0.50 | 0.50 | 0.50 | 0.50 |
| 28 | | | | | | | | |
| Mica, titanium | 0.30 | 0.30 | 0.30 | 0.30 | 0.30 | 0.30 | 0.30 | 0.30 |
| dioxide | | | | | | | | |
| Hydrolyzed | 0.50 | 0.50 | 0.50 | 0.50 | 0.50 | 0.50 | 0.50 | 0.50 |
| wheat protein | | | | | | | | |
| Fragrance | 1.25 | 1.25 | 1.25 | 1.25 | 1.25 | 1.25 | 1.25 | 1.25 |
| Ammonium | 7.00 | 7.00 | 7.00 | 7.00 | 7.00 | 7.00 | 7.00 | 7.00 |
| hydroxide | | | | | | | | |

*Hypnea Musciformis extract, Gellidiela Acerosa extract, Sargassum Filipendula extract, sorbitol.

The compositions were prepared by combining the ingredients and heating to 75° C. and mixing well. The mixture was cooled to 25° C.

### EXAMPLE 2

An developer composition was prepared as follows:

| Ingredient | % by weight |
|---|---|
| Water | QS |
| Methyl paraben | 0.05 |
| EDTA | 0.02 |
| Mineral oil | 0.60 |
| Cetearyl alcohol/ceteareth-20 (80:20) | 4.00 |
| Lauramide MEA | 0.50 |
| Cyclomethicone/trimethylsiloxysilicate (50:50) | 0.01 |
| Trimethylsilyl amodimethicone/C1-15 pareth-7/C12-16 | 2.00 |
| pareth-9/trideceth-12/glycerin/water (20:6:4:2:3:65) | |
| Hydrogen peroxide (35%) | 22.50 |
| Steareth-10 allyl ether/acrylates copolymer | 0.20 |
| Disodium phosphate | 0.03 |
| Phosphoric acid | 0.03 |

The composition was prepared by combining the ingredients, except for the hydrogen peroxide, at a temperature of 80° C. and mixing well. The mixture was cooled to 30° C. and the hydrogen peroxide was added and the mixture cooled to 25° C. The result was a 25 volume developer composition, e.g. containing 25 volume hydrogen peroxide.

### EXAMPLE 3

Aftercare hair conditioner compositions were prepared as follows:

| | | | | | Medium | |
|---|---|---|---|---|---|---|
| | Light Cool | No | Dark | Medium | Golden | Medium |
| | Brown | color | Brown | Auburn | Blonde | Brown |
| Ingredient | w/w % | | | | | |
| Water | QS | QS | QS | QS | QS | QS |
| Methyl paraben | 0.20 | 0.20 | 0.20 | 0.20 | 0.20 | 0.20 |
| Propyl paraben | 0.05 | 0.05 | 0.05 | 0.05 | 0.05 | 0.05 |
| Polyquaternium-10 | — | 0.20 | 0.20 | 0.20 | 0.20 | 0.20 |
| Panthenol | 0.01 | 0.01 | 0.01 | 0.01 | 0.01 | 0.01 |
| Behentrimonium chloride | 4.00 | 2.50 | 2.50 | 2.50 | 2.50 | 2.50 |
| Glycerin | 5.00 | 2.50 | 2.50 | 2.50 | 2.50 | 2.50 |
| Cetearyl alcohol | 6.00 | 5.00 | 5.00 | 5.00 | 5.00 | 5.00 |
| Cetyl alcohol | — | 1.00 | 1.00 | 1.00 | 1.00 | 1.00 |
| Dicaprylyl ether | — | 1.00 | 1.00 | 1.00 | 1.00 | 1.00 |
| Mango seed butter | 0.10 | 0.0001 | 0.0001 | 0.0001 | 0.0001 | 0.0001 |
| Jojoba seed oil | — | 0.20 | 0.20 | 0.20 | 0.20 | 0.20 |
| Amodimethicone, | 5.00 | 2.00 | 2.00 | 2.00 | 2.00 | 2.00 |
| trideceth-12, cetrimonium | | | | | | |
| chloride | | | | | | |
| Sodium benzotriazolyl | 0.005 | 0.001 | 0.001 | 0.001 | 0.001 | 0.001 |
| butylphenol sulfonate, | | | | | | |
| buteth-3, tributyl citrate | | | | | | |
| Fragrance | 0.50 | 0.50 | 0.50 | 0.50 | 0.50 | 0.50 |
| Cholesteryl oleyl | 0.01 | 0.0001 | 0.0001 | 0.0001 | 0.0001 | 0.0001 |
| carbonate, cholesteryl | | | | | | |
| chloride, cholesteryl | | | | | | |
| nonanoate | | | | | | |
| Isostearyl | 0.01 | 0.01 | 0.01 | 0.01 | 0.01 | 0.01 |
| citrate/glycolate/lactate/malate | | | | | | |
| Citric acid | 0.022 | 0.005 | 0.005 | 0.005 | 0.005 | 0.005 |
| Methylchloroisothiazolinone, | 0.04 | 0.04 | 0.04 | 0.04 | 0.04 | 0.04 |
| methylisothiazolinone | | | | | | |
| Polyethylene terephthalate, | 0.30 | 0.30 | 0.30 | 0.30 | 0.30 | 0.30 |
| acrylates copolymer | | | | | | |
| Basic Blue 99 | — | 0.36 | 0.005 | 0.010 | 0.0007 | 0.0225 |
| Basic Brown 17 | — | 0.08 | — | 0.008 | — | 0.005 |
| Basic Brown 16 | — | 0.28 | 0.06 | 0.006 | — | 0.005 |
| Basic Red 76 | — | 0.11 | 0.03 | — | 0.002 | 0.007 |
| Basic Yellow 57 | — | 0.22 | — | — | — | 0.0319 |

The hair conditioners were prepared by combining the oil phase and water phase ingredients separately, except for the dyes, then mixing them to emulsify at a temperature of 80-85° C. The mixture was cooled to 30° C. and the dyes were added. The mixture was cooled to 25° C.

### EXAMPLE 4

Aftercare shampoo compositions were prepared as follows:

| | Medium | Strawberry | |
|---|---|---|---|
| | Brown | Blonde | Strawberry |
| | w/w % | | |
| Water | QS | QS | QS |
| Magnesium aluminum silicate | 1.00 | 0.50 | 1.00 |
| Ethoxydiglycol | 5.00 | 5.00 | 5.00 |
| Methylparaben | 0.20 | 0.20 | 0.20 |
| Disodium EDTA | 0.05 | 0.05 | 0.05 |
| Erythorbic acid | 0.06 | 0.06 | 0.06 |
| HC Yellow No. 2 | 0.30 | 0.126 | 0.0315 |
| HC Yellow No. 4 | 0.308 | 0.330 | 0.0315 |
| HC Red No. 3 | 0.054 | 0.280 | 0.07 |
| HC Blue No. 2 | 1.18 | — | — |
| HC Red No. 13 | 0.183 | — | — |
| Disperse Orange 3 | — | 0.60 | 0.15 |
| Ethanolamine | 3.70 | — | — |
| Aminomethylpropanol | — | 3.6 | 3.6 |
| Oleic acid | 9.38 | 9.38 | 9.38 |
| Cetearyl alcohol | 3.00 | 3.00 | 3.00 |
| Emulsifying wax | 1.50 | 1.50 | 1.50 |
| Oleth-20 | 0.75 | 0.75 | 0.75 |
| Oleyl alcohol | 0.30 | 0.30 | 0.30 |
| Steareth-21 | 0.53 | 0.53 | 0.53 |
| Fragrance Oil | 0.50 | 0.50 | 0.50 |

The compositions were prepared by combining the ingredients and mixing well while heating to a temperature of about 75 to 80° C. The mixture was cooled to 25° C.

### EXAMPLE 5

Two sets of four swatches of 95% virgin gray hair, 1.2 grams, were tested for each of the oxidative hair color shades in Example 1. The first set of four swatches was colored with a mixture of 4.0 grams of the oxidative dye composition and 6.0 grams of developer for 10 minutes. The mixture was rinsed from the hair with water. Then 2.0 grams of the no color hair conditioner was applied to all four swatches for 2 minutes, then rinsed off with water for 30 seconds. The swatches were dried with a hair dryer. The second, third, and fourth swatches were washed 7, 14, and 21 times respectively, with Flex Extra Body Shampoo for 30 seconds, rinsed with water for 30 seconds. No color hair conditioner was applied to the respective swatches after 7, 14, and 21 shampoos for 2 minutes, then rinsing with water for 30 seconds. The swatches were dried with a hair dryer.

The second set of four swatches was colored with 4 grams of oxidative dye and 6.0 grams of developer for 10 minutes. The mixture was rinsed from the hair with water. Then 2.0 grams of the color hair conditioner was applied to all four swatches for 2 minutes, then rinsed off with water for 30 seconds. The swatches were dried with a hair dryer. The second, third, and fourth swatches from each set were washed 7, 14, and 21 times with Flex Extra Body Shampoo for 30 seconds and rinsed with water for 30 seconds. Color hair conditioner was applied for 2 minutes after 7, 14, and 21 shampoos respectively, rinsed off with water for 30 seconds, followed by drying with a hair dryer. The chromaticity of the swatches was measured using Datacolor Color Tools QC (version 1.2.1) spectrocolorimeter. The variables L, a, and b were measured where L is the level of lightness or darkness, a is the red and green components, and b is yellow and blue components, and the total change in color, ΔE is calculated as follows:

ΔE=\sqrt{(L−Lₒ)+(a−aₒ)²+(b−bₒ)²}

wherein L, a, and b are as above, and wherein the subscript o means prior to dyeing.

The results are as follows:

| | L | a | b | ΔL | ΔE |
|---|---|---|---|---|---|
| Dark Brown Oxidative Dye + | | | | | |
| No Color Hair Conditioner | | | | | |
| 0 shampoos + no color | 25.63 | 2.21 | 4.55 | | |
| conditioner | | | | | |
| 7 shampoos + no color | 28.62 | 2.35 | 5.66 | 3.12 | 3.15 |
| conditioner | | | | | |
| 14 shampoos + no color | 28.36 | 2.46 | 5.81 | 2.73 | 3.03 |
| conditioner | | | | | |
| 21 shampoos + no color | 28.53 | 2.57 | 6.46 | 2.89 | 3.52 |
| conditioner | | | | | |
| Dark Brown Oxidative Dye + | | | | | |
| Dark Brown Hair | | | | | |
| Conditioner | | | | | |
| 0 shampoos + no color | 25.63 | 2.21 | 4.55 | | |
| conditioner | | | | | |
| 0 shampoos + color | 24.04 | 2.40 | 5.37 | −1.59 | −2.10 |
| conditioner | | | | | |
| 7 shampoos + color | 25.50 | 2.73 | 5.76 | −0.13 | 1.35 |
| conditioner | | | | | |
| 14 shampoos + color | 25.25 | 2.84 | 6.24 | −0.38 | 1.99 |
| conditioner | | | | | |
| 21 shampoos + color | 25.51 | 2.94 | 6.41 | −0.13 | 2.02 |
| conditioner | | | | | |

The above results show that when the hair swatches are colored with oxidative dye and treated with no color hair conditioner right after the oxidative dye procedure, and then after 7, 14, and 21 shampoos, there is a more dramatic difference in L (lightness and darkness) and E (total color), e.g. the hair color washes out of the hair more readily. On the other hand, when the hair swatches are treated with dark brown oxidative dye followed by dark brown hair conditioner at 0, 7, 14, and 21 shampoos, the color is significantly more resistant to water wash out. The negative numbers mean that the hair becomes more colored after application of the aftercare conditioner.

| | L | a | b | ΔL | ΔE |
|---|---|---|---|---|---|
| Medium Auburn Oxidative | | | | | |
| Dye + No Color Hair | | | | | |
| Conditioner | | | | | |
| 0 shampoos + no color | 36.31 | 13.00 | 15.52 | | |
| conditioner | | | | | |
| 7 shampoos + no color | 37.45 | 12.21 | 15.16 | 1.14 | 1.48 |
| conditioner | | | | | |
| 14 shampoos + no color | 39.19 | 11.61 | 15.22 | 2.88 | 3.23 |
| conditioner | | | | | |
| 21 shampoos + no color | 39.47 | 11.50 | 15.33 | 3.16 | 3.52 |
| conditioner | | | | | |
| Medium Auburn Oxidative | | | | | |
| Dye + Medium Auburn Hair | | | | | |
| Conditioner | | | | | |
| 0 shampoos + no color | 36.31 | 13.00 | 15.52 | | |
| conditioner | | | | | |
| 0 shampoos + color | 35.09 | 15.45 | 16.17 | −1.23 | 3.39 |
| conditioner | | | | | |
| 7 shampoos + color | 36.43 | 14.85 | 15.55 | 0.11 | 2.37 |
| conditioner | | | | | |
| 14 shampoos + color | 36.38 | 15.25 | 15.66 | 0.06 | 2.37 |
| conditioner | | | | | |
| 21 shampoos + color | 36.84 | 15.32 | 15.94 | 0.53 | 2.50 |
| conditioner | | | | | |

The above results illustrate that the color is less resistant to water wash out in the hair treated with the color conditioner.

| | L | a | b | ΔL | ΔE |
|---|---|---|---|---|---|
| Light Cool Brown Oxidative | | | | | |
| Dye + No Color Hair | | | | | |
| Conditioner | | | | | |
| 0 shampoos + no color | 39.3 | 4.05 | 10.86 | | |
| conditioner | | | | | |
| 7 shampoos + no color | 42.35 | 3.62 | 11.08 | 3.06 | 3.11 |
| conditioner | | | | | |
| 14 shampoos + no color | 42.29 | 3.64 | 10.95 | 3.00 | 3.03 |
| conditioner | | | | | |
| 21 shampoos + no color | 42.79 | 3.61 | 11.31 | 3.49 | 3.55 |
| conditioner | | | | | |
| Light Cool Brown Oxidative | | | | | |
| Dye + Light Cool Brown | | | | | |
| Hair Conditioner | | | | | |
| 0 shampoos + no color | 39.3 | 4.05 | 10.86 | | |
| conditioner | | | | | |
| 0 shampoos + color | 36.22 | 4.79 | 11.21 | −3.07 | 3.34 |
| conditioner | | | | | |
| 7 shampoos + color | 38.56 | 5.00 | 11.63 | −0.74 | 2.10 |
| conditioner | | | | | |
| 14 shampoos + color | 39.77 | 4.94 | 11.63 | 0.47 | 2.27 |
| conditioner | | | | | |
| 21 shampoos + color | 40.2 | 4.98 | 11.71 | 0.91 | 2.46 |
| conditioner | | | | | |

The above results illustrate that the degree of color wash out seen in the hair treated with the color conditioner is less than what is seen in hair treated with no color conditioner. Further, in some cases, the hair becomes more colored after application of the color conditioner, as evidenced by the negative numbers in the ΔL column.

| | L | a | b | ΔL | ΔE |
|---|---|---|---|---|---|
| Medium Brown Oxidative | | | | | |
| Dye + No Color Hair | | | | | |
| Conditioner | | | | | |
| 0 shampoos + no color | 37.23 | 3.62 | 15.02 | | |
| conditioner | | | | | |
| 7 shampoos + no color | 39.89 | 3.68 | 11.40 | 2.67 | 4.52 |
| conditioner | | | | | |
| 14 shampoos + no color | 39.92 | 3.66 | 11.57 | 2.69 | 4.44 |
| conditioner | | | | | |
| 21 shampoos + no color | 40.87 | 3.83 | 11.98 | 3.64 | 4.88 |
| conditioner | | | | | |
| Medium Brown Oxidative | | | | | |
| Dye + Medium Brown Hair | | | | | |
| Conditioner | | | | | |
| 0 shampoos + no color | 37.23 | 3.62 | 15.02 | | |
| conditioner | | | | | |
| 0 shampoos + color | 36.83 | 3.86 | 15.38 | −0.40 | 1.44 |
| conditioner | | | | | |
| 7 shampoos + color | 38.35 | 4.38 | 12.08 | 1.12 | 3.85 |
| conditioner | | | | | |
| 14 shampoos + color | 37.93 | 4.54 | 12.12 | 0.71 | 3.52 |
| conditioner | | | | | |
| 21 shampoos + color | 37.68 | 4.83 | 12.67 | 0.45 | 2.93 |
| conditioner | | | | | |

The above results illustrate that when the oxidatively colored hair is treated with the color conditioner, the color wash out is significantly less.

### EXAMPLE 6

Gray hair swatches, 95% gray, 1.2 grams were used. The swatches were colored with a mixture of 4.0 grams of the oxidative dye composition of Example 1 and 6.0 grams of the developer composition of Example 2 for 10 minutes. The mixture was rinsed from the swatches with water for 30 seconds. The swatches were then shampooed with the compositions of Example 5 for 2 minutes then rinsed with water. The chromaticity of the swatches was measured using the same spectrocolorimeter as mentioned above. The results are as follows:

| | L | a | b | ΔL | ΔE |
|---|---|---|---|---|---|
| Ultra Light Natural Blonde | | | | | |
| Oxidative Dye + Strawberry | | | | | |
| Blonde Shampoo | | | | | |
| 1 color shampoo | 49.08 | 11.5 | 22.69 | | |
| 7 regular shampoos + 1 color | 47.08 | 13.74 | 25.79 | −2.01 | 4.32 |
| shampoo | | | | | |
| 24 shampoos regular | 46.45 | 15.13 | 26.39 | −2.63 | 5.81 |
| shampoos + 1 color shampoo | | | | | |
| Ultra Light Natural Blonde | | | | | |
| Oxidative Dye + Medium | | | | | |
| Brown Shampoo | | | | | |
| 1 color shampoo | 47.82 | 4.39 | 16.65 | | |
| 7 regular shampoos + 1 color | 40.03 | 4.9 | 13.53 | −7.79 | 8.41 |
| shampoo | | | | | |
| 24 regular shampoos + 1 color | 40.89 | 4.64 | 13.54 | −6.93 | 7.60 |
| shampoo | | | | | |

In the above example, the hair color shade itself is a high lift shade, which means that the hair is lightened only, providing a very slight change in tonality. The above results illustrate that the color shampoo actually applies more color to the hair as evidenced by the negative numbers in the ΔL column.

| | L | a | b | ΔL | ΔE |
|---|---|---|---|---|---|
| Dark Blonde Oxidative Dye + No | | | | | |
| color Hair Conditioner | | | | | |
| 0 shampoo + 1 no color | 34.95 | 6.91 | 11.86 | | |
| conditioner | | | | | |
| 7 regular shampoos + 1 no | 40.44 | 5.79 | 11.67 | 5.49 | 5.61 |
| color conditioner | | | | | |
| 24 regular shampoos + 1 no | 40.21 | 6.1 | 12.2 | 5.26 | 5.33 |
| color conditioner | | | | | |
| Dark Blonde Oxidative Dye + | | | | | |
| Medium Brown Shampoo | | | | | |
| 1 color shampoo | 33.38 | 6.39 | 11.78 | | |
| 7 regular shampoos + 1 color | 34.77 | 6.12 | 13.32 | 1.39 | 2.09 |
| shampoo | | | | | |
| 24 regular shampoos + 1 color | 34.72 | 5.47 | 12.47 | 1.34 | 1.76 |
| shampoo | | | | | |

The above results illustrate that the color fading is reduced when he color shampoo is used.

| | L | a | b | ΔL | ΔE |
|---|---|---|---|---|---|
| Medium Auburn Oxidative | | | | | |
| Dye + No Color Conditioner | | | | | |
| Initial | 36.31 | 13.00 | 15.52 | | |
| 7 regular shampoos + 1 no | 37.45 | 12.21 | 15.16 | 1.14 | 1.48 |
| color conditioner | | | | | |
| 14 shampoos + no color | 39.19 | 11.61 | 15.22 | 2.88 | 3.23 |
| conditioner | | | | | |
| 21 shampoos + no color | 39.47 | 11.50 | 15.33 | 3.16 | 3.52 |
| conditioner | | | | | |
| Medium Auburn Oxidative Dye + | | | | | |
| Medium Auburn shampoo | | | | | |
| Initial | 36.02 | 14.18 | 12.73 | | |
| Oxidative dye + medium | 34.92 | 14.21 | 13.32 | −1.1 | 1.25 |
| auburn shampoo | | | | | |
| 7 regular shampoos + 1 | 35.32 | 13.46 | 12.86 | −0.7 | 1.02 |
| medium auburn shampoo | | | | | |
| 24 regular shampoos + 1 | 38.5 | 12.95 | 13.3 | 2.48 | 2.82 |
| medium auburn shampoo | | | | | |

The above results illustrate that treating the hair with color conditioner and/or shampoo restores color to the hair.

### EXAMPLE 7

In a further study, the hair color fading seen with normal shampooing of the hair and the redeposit of color seen with use of the color conditioner was evaluated. Hair swatches, 95% gray, weighing about 1.5 grams each, were treated as follows in accordance with the procedures set forth in the previous examples:

| | L | a | b | ΔL | ΔE |
|---|---|---|---|---|---|
| Medium Golden Blonde | | | | | |
| Oxidative Dye + No Color | | | | | |
| Hair Conditioner | | | | | |
| 0 shampoos + no color | 58.11 | 5.40 | 18.68 | | |
| conditioner | | | | | |
| 7 shampoos + no color | 59.28 | 5.04 | 18.56 | 1.17 | 1.29 |
| conditioner | | | | | |
| 14 shampoos + no color | 59.78 | 4.78 | 18.36 | 1.66 | 1.86 |
| conditioner | | | | | |
| 21 shampoos + no color | 59.70 | 4.89 | 18.72 | 1.58 | 1.70 |
| conditioner | | | | | |
| Medium Golden Blonde | | | | | |
| Oxidative Dye + Medium | | | | | |
| Gold Blonde Hair Conditioner | | | | | |
| 0 shampoos + no color | 58.11 | 5.40 | 18.68 | | |
| conditioner | | | | | |
| 0 shampoos + color | 55.76 | 6.13 | 21.53 | −2.35 | 4.18 |
| conditioner | | | | | |
| 7 shampoos + color | 57.74 | 5.62 | 22.65 | −0.37 | 4.49 |
| conditioner | | | | | |
| 14 shampoos + color | 57.94 | 5.62 | 23.15 | −0.17 | 5.13 |
| conditioner | | | | | |
| 21 shampoos + color | 58.72 | 5.52 | 23.40 | 0.61 | 5.35 |
| conditioner | | | | | |

The above results show that the oxidative color is lost from the hair swatches after normal shampooing, and that the color is at least partially restored when the color shampoo is used between regular shampoos.

We claim:

1. A kit for oxidatively coloring hair and maintaining the color comprising, in combination:
(a) at least one oxidative composition,
(b) at least one developer composition that reacts with the oxidative composition to form a mixture that is capable of modifying the color of the hair; and
(c) at least one after care composition comprising one or more semi-permanent dyes selected to complement the color of the hair oxidatively colored by the mixture of compositions (a) and (b).

2. The kit of claim 1 wherein the oxidative composition and the developer composition are combined to produce color having Level 1 through Level 10, and of a specified tone, and the aftercare composition provides color that matches or complements the tone of the oxidatively colored hair.

3. The kit of claim 1 wherein the oxidative composition comprises at least one primary intermediate and at least one coupler for the formation of oxidative dye.

4. The kit of claim 3 wherein the oxidative composition additionally comprises one or more of a silicone, humectant, surfactant, conditioning agent, or mixtures thereof.

5. The kit of claim 1 wherein the oxidative composition is contained in a tube.

6. The kit of claim 1 wherein the developer composition is contained in a plastic bottle.

7. The kit of claim 1 wherein the developer composition comprises an aqueous solution or emulsion of hydrogen peroxide.

8. The kit of claim 7 wherein the developer composition is 25 volume or 30 volume hydrogen peroxide.

9. The kit of claim 1 wherein the aftercare composition is in the form of a hair conditioner.

10. The kit of claim 1 wherein the aftercare composition is in the form of a shampoo.

11. A method for improving color deposition and fade resistance of oxidatively colored hair by applying an after care composition containing at least one semi-permanent dye immediately after oxidatively coloring the hair, and repeating such applications of after care composition at intermittent time periods between oxidative dye sessions.

12. The method of claim 11 wherein the aftercare composition is a hair conditioner.

13. The method of claim 12 wherein the aftercare conditioner composition is in the form of a water and oil emulsion comprising about 0.0001-20% by weight of the total composition of one or more semi-permanent dyes.

14. The method of claim 13 wherein the semi-permanent dyes are basic dyes, disperse dyes, acid dyes, HC dyes, direct dyes, or mixtures thereof.

15. The composition of claim 14 wherein the semi-permanent dyes are basic dyes.

16. The method of claim 13 wherein the aftercare conditioner further comprises from about 0.001-5% of one or more cationic conditioning agents.

17. The method of claim 16 wherein the cationic conditioner agents are cationic quaternary ammonium compounds, cationic polymers or mixtures thereof.

18. The method of claim 11 wherein the aftercare composition is applied every one to thirty days.

19. The method of claim 11 wherein the aftercare composition has the same tone as the oxidative color applied to the hair.

20. The method of claim 11 wherein the aftercare composition has a different tone than the oxidative color applied to the hair.
